(19) United States
(12) Patent Application Publication
(10) Pub. No.: US 2002/0173486 A1
(43) Pub. Date: Nov. 21, 2002

(54) Methods for increasing bulk density of chondroitin

(75) Inventors: Nkere Kanu Ebube, Glen Allen, VA (US); William Antonio Mark, Mechanicsville, VA (US)

Correspondence Address: WYETH, PATENT LAW GROUP, FIVE GIRALDA FARMS, MADISON, NJ, 07940, US

(73) Assignee: American Home Products Corporation, Madison, NJ

(21) Appl. No.: 10/091,844

(22) Filed: Mar. 6, 2002

Related U.S. Application Data
(60) Provisional application No. 60/273,578, filed on Mar. 6, 2001.

(51) Int. Cl. A61K 31/737; B27N 3/00; A61K 31/7008

(52) U.S. Cl. 514/54; 514/62; 264/109

(57) ABSTRACT

The present invention relates to a method of increasing the bulk density of chondroitin, such as that derived from marine life, by roller compacting the chondroitin. The roller compacted chondroitin may be incorporated into a solid dosage form. The claimed method may also be applied to chondroitin/glucosamine mixtures.

### FIELD OF THE INVENTION

The present invention relates to methods for increasing the bulk density of chondroitin, such as that derived from marine life. The present invention also relates to compositions of such increased bulk density.

### BACKGROUND OF THE INVENTION

Chondroitin and glucosamine are chondroprotective agents which have found application in the treatment and/or prevention of osteoarthritis and related diseases of the joints. They are administered alone or in combination, normally in the form of a tablet or capsule.

Chondroitin has been reported to be effective in tissue repair and cartilage regeneration. Combinations of glucosamine and chondroitin are also effective in cartilage regeneration and joint maintenance. Chondroitin and glucosamine act by increasing chondrocyte anabolic activity and suppressing degradative action of mediators on cartilage. This facilitates natural tissue repair. See, e.g., H. Benedikt, Nat. Pharm., 1(8): 1, 22 (1997) and C. Bassleer, et al., Int. J. Tiss. Reac. XIV(5): 231-241: (1992). Additionally, chondroitin and glucosamine are believed to be safer and less toxic than steroids or non-steroidal anti-inflammatory drugs commonly administered to treat arthritis and related musculo-skeletal diseases.

The common forms of chondroitin are the sulfate and hydrochloride salts. Chondroitin sulfate is a soluble mucopolysaccharide derived from bovine, ovine or shark cartilage. Chondroitin sulfate derived from shark cartilage provides a rich, pure, and readily absorbed source of chondroitin.

The salt form of glucosamine facilitates its delivery and uptake by mammals and is commonly available as either hydrochloride (HCl) or sulfate (SO₄) salt. See, e.g., U.S. Pat. No. 5,364,845. The hydrochloride salt, which contains about 83% glucosamine, is richer in glucosamine than the sulfate salt, which contains only about 62% glucosamine. Glucosamine is commonly derived from mucopolysaccharides, mucoproteins and chitin.

However, the manufacture of solid dosage forms containing chodroitin and/or glucosamine has been problematic. Glucosamine HCl has poor compressibility, and chondroitin sulfate derived from marine life has low bulk density and poor flowability. Therefore, the amount of chondroitin and glucosamine which can be contained in a single tablet has been limited. Tablets have to be extremely large or a large number of tablets must be taken in order to administer effective amounts of the compounds.

Therefore, there is a need for dosage forms containing increased amounts of chondroitin through increased bulk density and methods for preparing the same. Higher density dosage forms of chondroitin would reduce the number of dosage units required to be administered to a patient and therefore increase patient compliance.

### OBJECTS OF THE INVENTION

It is an object of the invention to provide a method for increasing the bulk density of chondroitin, such as that derived from marine life. It is further an object of the invention to provide for compositions containing chondroitin having such increased bulk density.

### SUMMARY OF THE INVENTION

The invention relates to a method of increasing the bulk density of chondroitin or a mixture of chondroitin and glucosamine or a salt thereof by roller compacting the chondroitin or chondroitin/glucosamine mixture. The roller compacted chondroitin or chondroitin/glucosamine mixture may form or be incorporated into a solid dosage form, such as a tablet or capsule.

### BRIEF DESCRIPTION OF THE DRAWINGS

FIG. 1 is a graph of particle size versus the percentage of particles under a given particle size for glucosamine hydrochloride and a roller compacted 5:4 (by weight) mixture of glucosamine hydrochloride and chondroitin sulfate.

### DETAILED DESCRIPTION OF THE INVENTION

The present inventors have discovered that the bulk density of chondroitin can be increased by roller compaction. Roller compaction is a method of dry granulation. It is a continuous process and offers better control over processing parameters than, for example, slugging. Additionally, roller compaction does not require lubricants.

In roller compaction, a powder blend is agglomerated or laminated between pressurized rollers to form a ribbon or sheet of compact material. This compact ribbon is then milled to the desired granule size. Roller compaction has previously been utilized to densify and granulate a variety of pharmaceutical materials, such as aspirin, vitamin preparations, maltodextrin, microcrystalline cellulose, hydrous lactose, acetaminophen, and binary materials, such as aspirin and starch.

In the practice of the present invention, it has been found that the roller compaction process should expose the powdered chondroitin to pressures of at least about 500 psig. Preferably, such pressures should be in the range of about 800 to about 1500 psig. While operating temperatures are not critical to the practice of the present invention, high roller temperatures, which can result from high roller pressures and/or manufacturing throughput, should be avoided. If necessary, the raw material can be cooled. Alternatively, internally cooled rollers can be used.

The term “chondroitin” as used herein includes, but is not limited to, chondroitin and salts thereof, such as chondroitin sulfate and chondroitin hydrochloride. Chondroitin may be derived from animals, including, but not limited to, bovine, ovine, and marine life, such as shark cartilage.

The chondroitin used in roller compacting may be water soluble or insoluble. Since water-insoluble chondroitin sulfate typically has low bioavailability and efficacy, water-soluble chondroitin sulfate is preferred. Water soluble chondroitin sulfate is commercially available from Vanson, Inc. (Pfanstiehl Laboratory Inc.) of Waukegan, Ill. under the tradename of Polychon™ 60/40.

Typically, the bulk density of the roller compacted chondroitin, such as chondroitin sulfate, is at least about 0.37 g/mL. Preferably, the bulk density of the roller compacted chondroitin ranges from about 0.37 to about 0.6 g/mL. The density of marine-derived chondroitin sulfate which has not been treated in accordance with the method of the claimed invention is about 0.12 g/mL. Therefore, the densities resulting from the operation of the present invention are far higher, resulting in the ability to produce smaller tablets or incorporate a larger dosage into a similar number of equally-sized tablets.

Mixtures of chondroitin and glucosamine or salts thereof may also have their bulk densities increased through the application of the claimed method. This is unexpected since, as shown in the Comparative Examples hereof, the bulk density of glucosamine alone does not increase when subjected to roller compaction. Suitable salts of glucosamine include, but are not limited to, glucosamine hydrochloride and glucosamine sulfate. Glucosamine may be derived from mucopolysaccharides, mucoproteins, and chitin.

The weight ratio of glucosamine (or a salt thereof to chondroitin ranges broadly from about 1:100 to about 100:1. The typical mixture found in the compositions which are currently commercialized have a weight ratio of about 5:4. The preferred weight ratio of glucosamine (or a salt thereof) to chondroitin sulfate in the practice of the present invention is about 5:4. A preferred glucosamine is glucosamine hydrochloride, such as that produced by Wilke International of 15036 W. 106^{th} Street, Lexexa, Kans. 66215.

Generally, the bulk density of the roller compacted chondroitin/glucosamine mixture produced in the practice of the present invention is at least about 0.57 g/mL. Most preferably, chondroitin sulfate/glucosamine hydrochloride mixtures are employed. The bulk density of the chondroitin sulfate/glucosamine hydrochloride mixture preferably ranges from about 0.57 to about 0.68 g/mL.

Solid dosage forms of the roller compacted chondroitin may be prepared by tableting the roller compacted chondroitin through conventional tableting technology. While the amount of chondroitin in a given tablet is not critical, the use of the present invention allows for a substantial increase in such amount. In one embodiment of the present invention, the dosage form so produced contains about 600 mg of chondroitin sulfate. In another embodiment where a mixture of chondroitin sulfate and glucosamine (or a salt thereof) is employed, such dosage form contains about 600 mg of chondroitin sulfate and about 750 mg of glucosamine (or salt thereof).

Compositions containing chondroitin or chondroitin/glucosamine mixtures produced in accordance with the practice of the present invention may be formed into solid dosage forms such as tablets or caplets. Alternatively, the densified material produced herein may be inserted into capsules.

Such solid dosage forms may include other adjuvants as is known to those in the tableting and nutriceutical art. For instance, lubricants, antioxidants and/or flavoring may be included in the composition.

### DESCRIPTION OF THE PREFERRED EMBODIMENTS

The following examples illustrate the invention without limitations. All amounts are by weight unless otherwise specified.

### EXAMPLE 1

A 5:4 mixture (by weight) of glucosamine sulfate and chondroitin sulfate was prepared by mixing in a mixing bowl with a PK Blender (manufactured by Patterson & Kelly, a division of Harsco Corporation, East Stroudsburg, Pa. 18301) 300 grams of glucosamine hydrochloride (marketed by Pfanstiehl Laboratory Inc. of 1219 Glen Rock Avenue, Waukegan, Ill. 60085) and 240 grams of chondroitin sulfate (marketed under the tradename Polychon™ 60/40 by Vanson, Inc. of Waukegan, Ill.). The mixture was placed into the feed hopper of a Fitzpatric IR-520 chilsonator. The chilsonator converted the mixture into a compacted solid. The chilsonator was operated at a roll speed of 6 rpm, roll pressure of 1000 psig, vertical screw speed of 150 rpm, and horizontal screw speed of 15 rpm. The compact formed in the chilsonator was passed through a Fitzpatric M5A mill and the granulation collected. The mill was operated at a rotor speed of 300 rpm with a 4 bar rotor and a 0.050 inch rasping screen. The relative humidity was 57% and the temperature was 75° F.

The particle size of the granules was determined by sieve analysis. The bulk and tap densities and the Carr's index of the granules were also determined. Carr's index is a term commonly used to express the compressibility and flowability of powders. The lower the Carnr's index value, the better the flow of the powder. The results are shown in Table 1 below.

The particle size, bulk density, tap density, true density, and Carr's Index for untreated glucosamine hydrochloride, chondroitin sulfate, and blend of such glucosamine hydrochloride and chondroitin sulfate are also shown in Table 1 for comparison.

TABLE 1

| | | Bulk | Tap | True | |
|---|---|---|---|---|---|
| | Particle | Density | Density | Density | Carr's |
| Material | Size (m) | (g/mL) | (g/mL) | (g/mL) | Index (I) |
| Example 1 | 167.5 | 0.54 | 0.83 | — | 35.0 |
| Untreated | 6.83 | 0.13 | 0.28 | 1.52 | 53.6 |
| Chondroitin sulfate | | | | | |
| Untreated | 176 | 0.68 | 0.99 | 1.52 | 31.3 |
| Glucosamine HCl | | | | | |
| Blend of untreated | — | 0.27 | 0.47 | — | 42.6 |
| glucosamine HCl | | | | | |
| and chondroitin | | | | | |
| sulfate | | | | | |

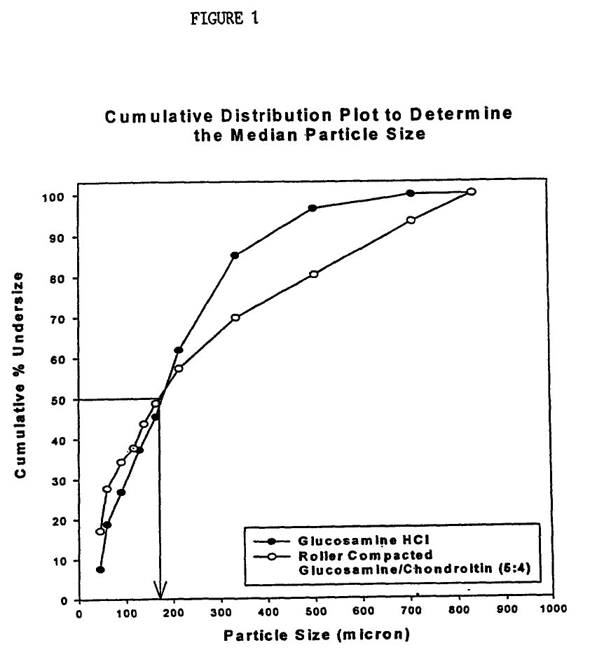

The particle size distribution (percentage of particles under a given particle size) in the roller compacted glucosamine hydrochloride/chondroitin sulfate is compared to untreated glucosamine hydrochloride in FIG. 1. This shows that was the particle size distribution of the powders was slightly altered following roller compaction, the median particle size of the glucosamine/chondroitin blend was approximately the same before and after roller compaction.

### EXAMPLE 2

The procedure in Example 1 was repeated, except that the roll pressure of the chilsonator was set at 1250 psig. The bulk density of the glucosamine hydrochloride/chondroitin sulfate mixture was found to be 0.57 g/mL, compared to a bulk density of 0.27 g/mL for the untreated mixture and a bulk density of 0.54 g/mL for the material processed in accordance with the parameters set forth in Example 1. This mixture further was found to possess a Carr's Index value of 31.3.

### EXAMPLE 3

The procedure in Example 1 was repeated with only chondroitin sulfate, rather than the glucosamine hydrochloride/chondroitin sulfate mixtures used in Examples 1 and 2. The chilsonator was operated at a roll speed of 6 rpm, roll pressure of 750 psig, vertical screw speed of 200 rpm, and horizontal screw speed of 42 rpm. The bulk density of the granules was 0.37 g/mL, which is nearly triple the bulk density of untreated chondroitin sulfate, which has a bulk density of 0.13 g/mL.

### COMPARATIVE EXAMPLE 4

The procedure in Example 1 was repeated with glucosamine hydrochloride in lieu of the glucosamine hydrochloride/chondroitin sulfate mixture. The chilsonator was operated at a roll speed of 6 rpm, roll pressure of 1300 psig, vertical screw speed of 150 rpm, and horizontal screw speed of 8 rpm. The treated material was found to possess a bulk density of 0.68 g/mL. This represents no change in bulk density relative to the unprocessed glucosamine hydrochloride.

### COMPARATIVE EXAMPLE 5

The procedure of Example 4 was repeated with the chilsonator operated at a roll speed of 6 rpm, roll pressure of 1500 psig, vertical screw speed of 150 rpm, and horizontal screw speed of 8 rpm. The treated material was found to possess a bulk density of 0.68 g/mL. Again, this represents no change in bulk density relative to the unprocessed glucosamine hydrochloride.

All patents, publications, applications, and test methods mentioned herein are hereby incorporated by reference.

Many variations of the present invention will suggest themselves to those skilled in the art in light of the above, detailed description. All such obvious variations are within the full intended scope of the appended claims.

What is claimed is:

1. A method of increasing the bulk density of chondroitin, said method comprising roller compacting said chondroitin at pressures of at least 500 psig.

2. The method as defined in claim 1, wherein the roller compaction is conducted at pressures ranging from about 800 to about 1500 psig.

3. The method as defined in claim 1, wherein the chondroitin is selected from the group consisting of chondroitin sulfate, chondroitin hydrochloride and mixtures thereof.

4. The method as defined in claim 3, wherein the chondroitin is chondroitin sulfate.

5. The method as defined in claim 1, wherein the chondroitin is derived from marine life.

6. The method as defined in claim 5, wherein the chondroitin is derived from shark cartilage.

7. The method as defined in claim 1, wherein the bulk density of said roller compacted chondroitin is at least 0.37 g/mL.

8. The method as defined in claim 1, wherein the bulk density of said roller compacted chondroitin ranges from about 0.37 to about 0.6 g/mL.

9. A method of increasing the bulk density of a mixture of chondroitin and glucosamine (or salts thereof), said method comprising roller compacting said mixture at pressures of at least 500 psig.

10. The method as defined in claim 9, wherein the roller compaction is conducted at pressures ranging from about 800 to about 1500 psig.

11. The method as defined in claim 9, wherein the ratio of chondroitin to glucosamine ranges from about 100:1 to about 1:100.

12. The method as defined in claim 9, wherein the ratio of chondroitin glucosamine is about 4:5.

13. The method as defined in claim 9, wherein the chondroitin is selected from the group consisting of chondroitin sulfate, chondroitin hydrochloride and mixtures thereof.

14. The method as defined in claim 13, wherein the chondroitin is chondroitin sulfate.

15. The method as defined in claim 9, wherein the chondroitin is derived from marine life.

16. The method as defined in claim 15, wherein the chondroitin is derived from shark cartilage.

17. The method as defined in claim 9, wherein the salt of glucosamine is selected from the group consisting of glucosamine sulfate, glucosamine hydrochloride and mixtures thereof.

18. The method as defined in claim 17, wherein the salt of glucosamine is glucosamine hydrochloride.

19. The method as defined in claim 9, wherein the bulk density of said roller compacted mixture is at least about 0.57 g/mL.

20. The method as defined in claim 19, wherein the bulk density of said roller compacted mixture ranges from about 0.57 to about 0.68 g/mL.

21. The method as defined in claim 20, wherein the salt of glucosamine is glucosamine hydrochloride and the chondroitin is chondroitin sulfate.

22. The method as defined in claim 21, wherein the bulk density of said roller compacted mixture is at least about 0.57 g/mL.

23. The method as defined in claim 22, wherein the bulk density of said roller compacted mixture ranges from about 0.57 to about 0.68 g/mL.

24. A method of preparing a dosage form containing chondroitin, said method comprising:
(A) roller compacting chondroitin at pressures of at least 500 psig, and
(B) tableting said roller compacted chondroitin.

25. The method as defined in claim 24, wherein the roller compaction is conducted at pressures ranging from about 800 to about 1500 psig.

26. The method as defined in claim 24, wherein the chondroitin is selected from the group consisting of chondroitin sulfate, chondroitin hydrochloride and mixtures thereof.

27. The method as defined in claim 26, wherein the chondroitin is chondroitin sulfate.

28. The method as defined in claim 24, wherein the chondroitin is derived from marine life.

29. The method as defined in claim 28, wherein the chondroitin is derived from shark cartilage.

30. The method as defined in claim 26, wherein the bulk density of said roller compacted chondroitin is at least 0.37 g/mL.

31. The method as defined in claim 26, wherein the bulk density of said roller compacted chondroitin ranges from about 0.37 to about 0.6 g/mL.

32. A method of preparing a dosage form of a mixture of chondroitin and glucosamine or a salt thereof, said method comprising:
(A) roller compacting a mixture of chondroitin and glucosamine or a salt thereof at pressures of at least 500 psig., and
(B) tableting said roller compacted mixture.

33. The method as defined in claim 32, wherein the roller compaction is conducted at pressures ranging from about 800 to about 1500 psig.

34. The method as defined in claim 32, wherein the ratio of chondroitin to glucosamine ranges from about 100:1 to about 1:100.

35. The method as defined in claim 34, wherein the ratio of chondroitin to glucosamine is about 4:5.

36. The method as defined in claim 32, wherein the chondroitin is selected from the group consisting of chondroitin sulfate, chondroitin hydrochloride and mixtures thereof.

37. The method as defined in claim 36, wherein the chondroitin is chondroitin sulfate.

38. The method as defined in claim 32, wherein the chondroitin is derived from marine life.

39. The method as defined in claim 32, wherein the chondroitin is derived from shark cartilage.

40. The method as defined in claim 32, wherein the salt of glucosamine is selected from the group consisting of glucosamine sulfate, glucosamine hydrochloride and mixtures thereof.

41. The method as defined in claim 40, wherein the glucosamine is glucosamine hydrochloride.

42. The method as defined in claim 32, wherein the bulk density of said roller compacted mixture is at least about 0.57 g/mL.

43. The method as defined in claim 42, wherein the bulk density of said roller compacted mixture ranges from about 0.57 to about 0.68 g/mL.

44. The method as defined in claim 32, wherein the salt of glucosamine is glucosamine hydrochloride and the chondroitin is chondroitin sulfate.

45. The method as defined in claim 44, wherein the bulk density of said roller compacted mixture is at least about 0.57 g/mL.

46. The method as defined in claim 45, wherein the bulk density of said roller compacted mixture ranges from about 0.57 to about 0.68 g/mL.

47. A solid dosage form comprising tableted, roller compacted chondroitin.

48. The dosage form as defined in claim 47, wherein the amount of chondroitin in said dosage form is about 600 mg.

49. The dosage form as defined in claim 47, wherein the chondroitin is chondroitin sulfate.

50. The dosage form as defined in claim 47, wherein the bulk density of said roller compacted chondroitin is at least about 0.37 g/mL.

51. The dosage form as defined in claim 50, wherein the bulk density of said roller compacted chondroitin ranges from about 0.37 to about 0.6 g/mL.

52. A solid dosage form comprising a tableted, roller compacted mixture of chondroitin and glucosamine or a salt thereof.

53. The dosage form as defined in claim 52, wherein the ratio of chondroitin to glucosamine ranges from about 100:1 to about 1:100.

54. The dosage form as defined in claim 52, wherein the ratio of chondroitin to glucosamine is about 4:5.

55. The dosage form as defined in claim 52, wherein the chondroitin is selected from the group consisting of chondroitin sulfate, chondroitin hydrochloride and mixtures thereof.

56. The dosage form as defined in claim 52, wherein the salt of glucosamine is selected from the group consisting of glucosamine sulfate, glucosamine hydrochloride and mixtures thereof.

57. The dosage form as defined in claim 56, wherein the salt of glucosamine is glucosamine hydrochloride.

58. The dosage form as defined in claim 52, wherein the bulk density of said roller compacted mixture is at least about 0.57 g/mL.

59. The dosage form as defined in claim 58, wherein the bulk density of said roller compacted mixture ranges from about 0.57 to about 0.68 g/mL.

60. The dosage form as defined in claim 52, wherein the salt of glucosamine is glucosamine hydrochloride and the chondroitin is chondroitin sulfate.

61. The dosage form as defined in claim 60, wherein the bulk density of said roller compacted mixture is at least about 0.57 g/mL.

62. The dosage form as defined in claim 60, wherein the bulk density of said roller compacted mixture ranges from about 0.57 to about 0.68 g/mL.

63. The dosage form as defined in claim 52, wherein the amount of chondroitin sulfate in said dosage form is about 600 mg and the amount of glucosamine or salt thereof is about 750 mg.
